(19) United States
(12) Patent Application Publication
(10) Pub. No.: US 2011/0059533 A1
(43) Pub. Date: Mar. 10, 2011

(54) FLUORESCENCE DETECTION SYSTEM, METHOD, AND DEVICE FOR MEASURING BIOMOLECULES

(76) Inventors: Yue-Ming HSIN, Tainan City (TW); Chun-Yu Liao, Taipei City (TW)

(21) Appl. No.: 12/711,918

(22) Filed: Feb. 24, 2010

(30) Foreign Application Priority Data
Sep. 7, 2009 (TW) 098130051

(51) Int. Cl. G01N 33/52; G01N 33/50; G01J 1/58; G01N 21/85

(52) U.S. Cl. 436/71; 436/94; 436/86; 436/172; 250/458.1; 250/573; 250/200

(57) ABSTRACT

A fluorescence detection system for measuring biomolecules is disclosed, which includes a fluorescence detection device, a light source, a sample-loading unit, and an analysis-reading device. The fluorescence detection device has a substrate and plural phototransistors arranged on the substrate, and each phototransistor contains an emitter, a collector locating on the substrate, and a base between the emitter and the collector. The base-collector diode junction functions as an absorber to convert fluorescence to photocurrent. The light source serves to excite a fluorescent dye contained in a biomolecule sample. The sample-loading unit is used to load or transport the excited biomolecule sample onto a sensing zone of the fluorescence detection device. The analysis-reading device is to measure photocurrent output from the fluorescence detection device under a bias. Hence, the biomolecule content can be easily determined by the fluorescence detection system.

### BACKGROUND OF THE INVENTION

1. Field of the Invention

The present invention relates to a fluorescence detection system, method, and device for measuring biomolecules and, more particularly, to a fluorescence detection system, method, and device combined with optoelectronic semiconductor for measuring biomolecules.

2. Description of Related Art

In clinical medicine, each organ of a human body can be preliminarily estimated for its medical situation by detecting changes of corresponding biomolecules such as glucoses and proteins in blood and urine. For example, in clinical detection of nephropathy, whether glomeruli function well can be examined by quantification of urinary proteins.

Among conventional measurements of urinary proteins, one method for qualitative analysis is to use indicator papers. However, a false positive or negative may be demonstrated on the indicator papers, resulting in faulty identification. Besides, turbidimetric immunoassay, high-pressure liquid chromatography, and fluorescence detection are other methods which can be utilized for quantitative analysis. The former two methods can precisely measure the protein amount, but they require complicated operation and, expensive equipment and reagents. Likewise, the last method needs to be performed with complex optical instruments as well as optical signal analysis software. Hence, the aforesaid three methods consume too much time and money and are not convenient to whole processes for detection.

Therefore, it is desirable to develop high-sensitivity, high-accuracy, compact-size, and low-cost biosensors for detecting specific biomolecules and promptly affording the subsequent results without much time being spent. Accordingly, patients will not have to spend time in detailed examination at hospital and can preliminarily examine themselves so as to achieve prevention of those targeted medical conditions.

### SUMMARY OF THE INVENTION

In one aspect of the present invention, there is provided a fluorescence detection system for measuring biomolecules, which includes a fluorescence detection device comprising a substrate and plural phototransistors arranged on the substrate, wherein each phototransistor comprises an emitter, a collector locating on the substrate, and a base between the emitter and the collector, and a base-collector diode junction functions as an absorber to convert fluorescence to photocurrent; a light source exciting a fluorescent dye contained in a biomolecule sample; a sample-loading unit loading or transporting the biomolecule sample containing the excited fluorescent dye onto a sensing zone of the fluorescence detection device; and an analysis-reading device measuring photocurrent output from the fluorescence detection device under a bias. In the fluorescence detection system, the analysis-reading device can further comprise a computation module calculating a biomolecule content of the biomolecule sample from the photocurrent. The analysis-reading device can also functions as a transporter to transport the biomolecule sample through the sensing zone of the fluorescence detection device.

In another aspect of the present invention, there is provided a fluorescence detection method, which includes the following steps: illuminating a biomolecule sample containing a fluorescent dye by a light source; detecting the biomolecule sample by a fluorescence detection device under a bias, wherein the fluorescence detection device comprises a substrate and plural phototransistors arranged on the substrate, and each phototransistor comprises an emitter, a collector locating on the substrate, and a base between the emitter and the collector, wherein a base-collector diode junction functions as an absorber to convert fluorescence to photocurrent; and measuring photocurrent output from the fluorescence detection device. The fluorescence detection method can further comprise the following step: converting the photocurrent into a biomolecule content of the biomolecule sample based on a current-content standard curve.

In further another aspect of the present invention, there is provided a fluorescence detection device for measuring biomolecules comprising: a substrate; and plural phototransistors arranged on the substrate, wherein each phototransistor comprises an emitter, a collector locating on the substrate, and a base between the emitter and the collector, and a base-collector diode junction functions as an absorber to convert fluorescence to photocurrent. Actually, when consumers use the fluorescence detection device to measure biomolecules at home, any natural or interior light can directly serve as the light source for exciting the fluorescence dye. Once fluorescence is emitted from the excited fluorescent dye, the detection device can detect the fluorescence and then output the result of the measurement. Hence, the detection device of the present invention has the advantage of low price and can be operated easily without a specific exciting light source provided therein.

The bias applied in the fluorescence detection device can vary according to materials used in the fluorescence detection device and the total number of the phototransistors. Hence, the bias is not particularly limited as long as the measurable photocurrent output by the fluorescence detection device can be detected by the analysis-reading device. Preferably, the bias applied is in a range that the biomolecule content is proportional to the photocurrent. For example, the bias can be in a range of 0.5 to 50 V, and preferably in a range of 1 to 10 V.

In the aforesaid fluorescence detection device, if the area of the emitter in each phototransistor is smaller than that of the base, the phototransistor has the larger base beneficial for fluorescence absorption. The partial or total parallel connection of the phototransistors can afford enhanced photocurrent. In addition, the phototransistors can be arranged in a matrix to make the layout focus and be integrated in a compact size. The material system of the emitter, the collector, and the base in the phototransistors is not limited. For example, at least one of AlGaAs/GaAs, InGaP/GaAs, AlInAs/InGaAs/InP, InP/InGaAs, InP/GaAsSb/InP, AlInAs/GaAsSb/InP, Si/SiGe, and GaN/SiC can be used as the material system.

The light source functions as a provider of excitation light. The excitation light excites the fluorescent dye, which is bound on the biomolecule, into excited state. Accordingly, the kind of the light source requires to be chosen according to the kind of the fluorescent dye. For example, when the fluorescent dye, IR-783, is utilized, red, white or infrared LEDs can be applied to excite IR-783 into an excited state. As to the illumination time of the biomolecule sample, the kind of the fluorescent dye, the fluorescence detection device, the wavelength and intensity of the light source all are related. Basically, the illumination time is supposed to be as short as possible so that the time consumed in the measurement of the biomolecule, can be reduced to a minimum. For example, if IR-783 is applied, the time illuminated by white LEDs can be in a range of 30 sec to 30 min, and preferably in a range of 5 to 15 min.

Referring to the fluorescent dye, the kind of the biomolecule can be based in order to select a fluorescent dye specifically bound to the biomolecule. For instance, when human serum albumin (HSA) is a target of detection, IR-783 can be used as a fluorescent dye due to its specificity to human serum albumin.

In the fluorescence detection device, system, and method mentioned above, the biomolecule is not limited to a particular kind as long as the suitable fluorescent dye and photoelectric material system can be found. Hence, any nucleic acid, carbohydrate, protein, lipid, phospholipid, glycolipid, sterol, vitamin, hormone, amino acid, nucleotide, peptide and so forth can be a suitable target of detection.

In conclusion, the present invention uses a fluorescent dye specifically bound to the tested sample and then selects a light source according to the fluorescent dye. For example, when IR-783 is used, visible light is selected to accomplish excitation of IR-783. Once the fluorescent dye absorbs the light energy from the light source and then emits the light with the wavelength in a range absorbable by the phototransistors, the phototransistors can convert the absorbed light into a photocurrent. Accordingly, the biomolecule content of the tested sample can be recognized. In other words, two techniques, phototransistors and fluorescent reaction, are combined in the present invention to provide a fluorescence detection system, method, and device for measuring biomolecules. Particularly, the present invention has excellent sensitivity to low concentration of the biomolecule, and can real-timely and promptly monitor the concentration change of the biomolecule. In contrast to the conventional fluorescence detection with complicated instructions, the present invention possesses an advantage of the prompt detection of the biomolecule.

Other objects, advantages, and novel features of the invention will become more apparent from the following detailed description when taken in conjunction with the accompanying drawings.

### BRIEF DESCRIPTION OF THE DRAWINGS

FIG. 1 is a cross-sectional view of a phototransistor in Example 1 of the present invention;

FIG. 2 shows a layout of two phototransistors connected in parallel in Example 1 of the present invention;

FIG. 3 is a standard curve of current vs. HSA concentration in Example 2 of the present invention;

FIG. 4 shows a configuration of a fluorescence detection system in Example 3 of the present invention; and

FIG. 5 is a standard curve of current vs. HSA concentration in Example 3 of the present invention.

### DETAILED DESCRIPTION OF THE PREFERRED EMBODIMENT

The fluorescence detection system, method, and device for measuring biomolecules disclosed in the present invention combines phototransistors with fluorescent reaction, and then detects photocurrent induced by the biomolecule sample to promptly identify the biomolecule content.

The fluorescence detection device of the present invention comprises multiple phototransistors. However, the total number of the phototransistors is not limited, and can be determined according to client demands. For instance, 10, 20, 40, 80, 200, 400, 800, 1000, and even more phototransistors can be the total number. In addition, the phototransistors can be connected partially or totally in parallel, and also can be arranged in an array.

In the present invention, a suitable fluorescent dye can be selected according to the kind of tested biomolecules, the material system of the phototransistors, and so forth. For example, in regard to DNA detection, ethidium bromide (EtBr) can be used as a fluorescent dye. When EtBr is inserted into DNA, EtBr can fluoresce after being excited by ultraviolet light. Other fluorescent dyes, such as SYTOX Blue, SYTOX Green, SYTOX Orange, Acridine Orange and LDS 751, also can be bound to DNA, and fluoresce after each being excited by light sources having a wavelength in a specific range. Referring to protein detection, such as HSA, a specific fluorescent dye IR-783 can be used. Besides, as to glucose or other biomolecules, one skilled in the art of the present invention can easily understand how to select a suitable fluorescent dye for the biomolecules.

Since different material systems of the phototransistors have their specific range of the absorbable light wavelength and corresponding absorption coefficient, the kind of the fluorescent dyes should be chosen in consideration of the kind of the biomolecules as well as whether the fluorescence emitted from the fluorescent dyes is absorbable by the material systems of the phototransistors, and thereby converted into photocurrent. Therefore, the material system of the base and the collector in each phototransistor of the present invention should be applied together with a suitable fluorescent dye.

The light source utilized in the present invention can have any power and wavelength without limitation. A suitable light source can be selected based on the used kind of the fluorescent dyes. For example, an infrared LED having power of −32 to −50 dBm and wavelength between 790 to 900 nm, a red LED having power of −35 to −70 dBm and wavelength between 605 to 735 nm, and a white LED having power of −33 to −65 dBm and wavelength between 400 to 850 nm, can each be applied in the present invention.

Because of the specific embodiments illustrating the practice of the present invention, one skilled in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

The drawings of the embodiments in the present invention are all simplified charts or views, and only reveal elements relative to the present invention. The elements revealed in the drawings are not necessarily aspects of the practice, and quantity and shape thereof are optionally designed. Further, the design aspect of the elements can be more complex.

### Example 1

### Fluorescence Detection Device

First, a wafer mainly made of AlGaAs/GaAs and purchased from KOPIN, was prepared. After the wafer was washed, it was processed by photolithography and wet etching repeatedly to define emitter, base, and collector mesa areas, and emitter and collector circuit regions, and by vapor deposition to form emitter and collector metal electrodes and metal circuits thereof (made of Ni, Ge, Au, Ti, Al or a combination thereof). Moreover, high-temperature annealing was performed to allow good ohmic contact between the metal and the semiconductor. Finally, a passivation layer was deposited to protect a resultant phototransistor with NPN heterojunction. Sixty phototransistors obtained by the abovementioned were arranged in an array, and connected in parallel via metal circuits made by photolithography combined with vapor deposition so as to afford a fluorescence detection device.

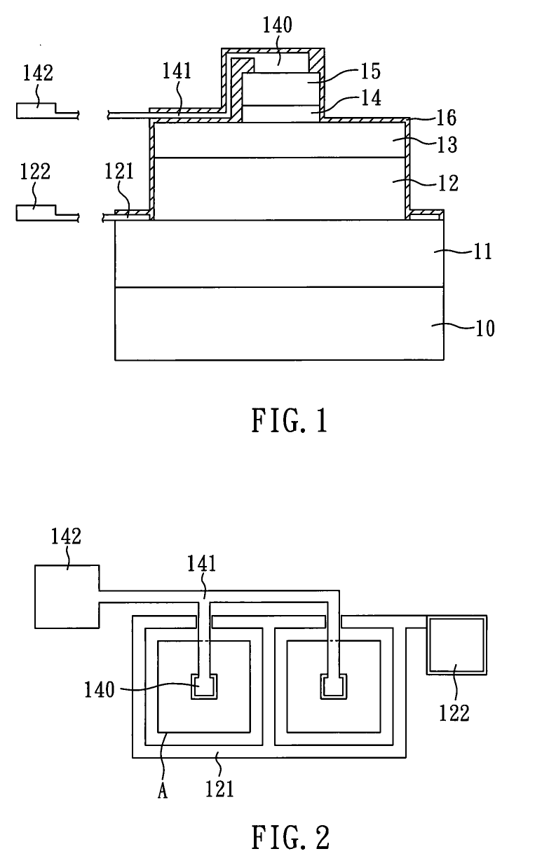

FIG. 1 shows a cross-sectional view of the phototransistor manufactured above. As shown in FIG. 1, sub-collector 11 locates on a substrate 10. A collector 12 and a collector metal circuit 121 both locate on the sub-collector 11. The collector metal circuit 121 surrounds the collector 12 and is electrically connected to the sub-collector 11. In addition, a base 13 locates on the collector 12. Since the base 13 is supplied with current converted from fluorescence in the present invention, there is no need to construct a metal electrode for the base 13. Furthermore, an emitter 14 locates on the base 13 and is of a smaller area than the base 13. Thus, this phototransistor has the extensive base 13 advantageous to fluorescence absorption to promote sensitivity thereof. An emitter cap 15 locates between the emitter and an emitter metal electrode 140, and an emitter metal circuit 141 is connected to the emitter metal electrode 140. The emitter metal circuit 141 is embedded in a passivation layer 16. The passivation layer 16 separates the collector metal circuits 121 from the collector 12 and covers the exposed collector metal circuits 121, collector 12, base 13, emitter 14, emitter cap 15, and emitter metal electrode 140 for insulating protection of the phototransistor.

FIG. 2 shows a circuit layout of two phototransistors connected in parallel of sixty phototransistors arranged in an array. As shown in FIG. 2, two neighboring phototransistors are connected in parallel via the collector metal circuit 121 and the emitter metal circuit 141, and the collecting locations of the parallel connection of the emitter and collector metal circuits are respectively defined as a collector electrode pad 122 and an emitter electrode pad 142. The collector and emitter electrode pads 122, 142 are used to connect positive and negative electrodes of a power supply, respectively. Accordingly, a suitable bias can be applied on the collector 12 and the emitter 14, respectively. Zone A is the area where phototransistor detects fluorescence of a sample.

### Example 2

### HSA Assay

Na₂HPO₄ buffer (10 mM) was prepared, and its pH value was adjusted to 7.4 by phosphoric acid. HSA solution was diluted with the Na₂HPO₄ buffer to the concentrations of 0.01, 0.03, 0.05, and 0.07 mg/mL.

An infrared fluorescent dye IR-783 (C₃₈H₄₆CIN₂NaO₆S₂, shown as the following formula) purchased from Sigma Aldrich was dissolved in a small amount of methanol, and then diluted with the Na₂HPO₄ buffer to the concentration of 0.02 mg/mL. When IR-783 (specific to HSA) is bound to HSA, IR-783 achieves chemically stable state. Once IR-783 in this state is excited by light, IR-783 absorbs the light, is converted into the excited state, and then fluoresces in a spectrum of 750 to 850 nm. This spectrum accords with the range of the light wavelength that is absorbable by the base (GaAs) of each phototransistor in the fluorescence detection device of Example 1. Hence, IR-783 is suitable for being applied in the fluorescence detection device of Example 1.

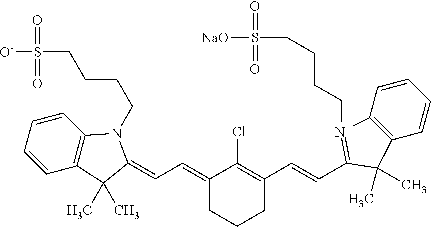

A semiconductor device analyzer (B1500A, Agilent) was connected to a probe station on which the fluorescence detection device of Example 1 was put.

Tested HSA solutions with different concentrations respectively were mixed with equal volume of the IR-783 solution prepared above, and illuminated by infrared LED (power: −32 to −50 dBm, emission wavelength: 790 to 900 nm) for five minutes. Then, 1 μL of each mixed solution was sucked by a pipette on the zone A of the phototransistors in the fluorescence detection device of Example 1, and retained in the dark for 30 sec in order to prevent any slight error. The semiconductor device analyzer provided a bias of 1.0 V to the fluorescence detection device, and meantime collected photocurrent signals output from the fluorescence detection device.

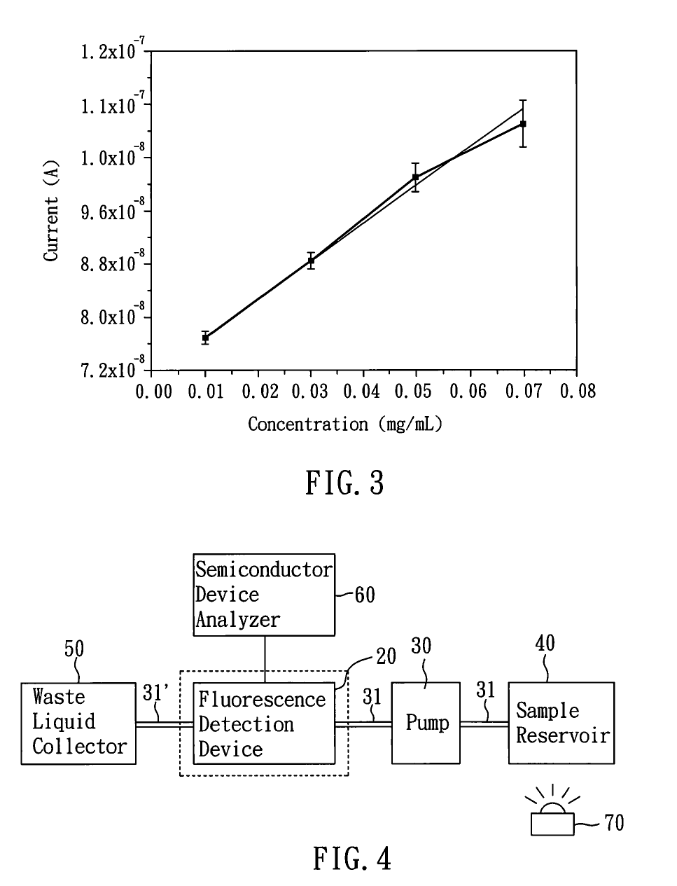

FIG. 3 demonstrates the result, where a straight line obtained shows that the photocurrent is proportional to the HSA concentration between 0.01 and 0.07 mg/mL, and the equation of the straight line is Y=7.13×10⁻⁸+5.72×10⁻¹⁰ X in which Y represents the photocurrent in a unit of ampere (A) and X represents the HSA concentration in a unit of μg/mL. The result indicates that the photocurrent increase 0.572 nA as the HSA concentration increases 1 μg/mL between 0.01 and 0.07 mg/mL of the HSA concentration if the measurement is carried out by the fluorescence detection device of Example 1.

Hence, after a current-concentration standard curve is obtained, the fluorescence detection device of the present invention can cooperate with fluorescent reaction to identify an unknown concentration of an HSA solution. Using the photocurrent output from the fluorescence detection device, a corresponding concentration of the HSA solution can be recognized according to the current-concentration standard curve.

### Example 3

### Fluorescence Detection System

The phototransistors used in the present example were prepared in the same manner of Example 1. The fluorescence detection device of the present example was configured with 808 phototransistors connected in parallel, and mounted on a printed circuit board (PCB). Electrode pads of the fluorescence detection device were connected to metal circuits of the PCB by a wire-bonding machine.

FIG. 4 shows a configuration of the fluorescence detection system. With reference to FIG. 4, a sample reservoir 40, a pump 30 (BT-1002J, Baoding Longer Precision Pump Co., Ltd.), a light source 70, a waste liquid collector 50, and a semiconductor device analyzer 60 were prepared. HSA solution was transported by the pump 30 from the sample reservoir 40 via a tube (2 mm) onto the sensing zone of the fluorescence detection device 20, and then to the waste liquid collector 50 via another tube 31′. Besides, the fluorescence detection device 20 was connected to the semiconductor device analyzer 60 by a solid wire. The pump 30 and the tubes 31 and 31′ functioned as a sample-loading unit to transport or load a biomolecule solution.

In the measurement of the HSA solution, the HSA solutions prepared in Example 1 were mixed with equal volume of the IR-783 solution, and then poured into the sample reservoir 40. In the present example, an infrared LED was used as the light source 70 to illuminate the solution in the sample reservoir 40, and then the illuminated solution was transported by the pump 30 via the tube 31 onto the sensing zone of the fluorescence detection device 20. Meanwhile, the semiconductor device analyzer 60 provided a bias of 1V to the fluorescence detection device 20, and collected photocurrent signals output from the fluorescence detection device 20.

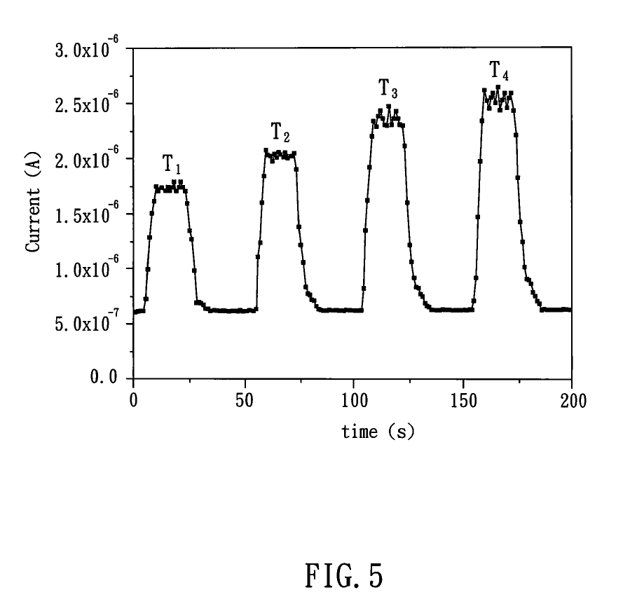

The four different concentrations (0.01, 0.03, 0.05, and 0.07 mg/mL) of the HSA solutions were measured in order, and the tubes were washed with pure water between two measurements. FIG. 5 shows the result, i.e. a current-time curve.

In FIG. 5, the initial several seconds exhibit the dark current which means the tested solution had not entered the fluorescence detection system yet. The time zone T₁ is the period when the HSA solution of 0.01 mg/mL was detected. During the time zone T₁, the photocurrent was kept stable in a range. After the pure water was loaded into the tubes for washing, the photocurrent dramatically dropped to the initial dark current. Then, the solutions of 0.03, 0.05, and 0.07 mg/mL were detected in sequence. The time zones T₂, T₃, and T₄ shown in FIG. 5 represent the periods when the HSA solutions of 0.03, 0.05, and 0.07 mg/mL were detected, respectively. In addition, between two measurement operations the tubes were washed with pure water. The photocurrent values of the HSA solutions (0.01, 0.03, 0.05, and 0.07 mg/mL) obtained from the time zones T₁, T₂, T₃, and T₄ were analyzed by linear regression. As shown in FIG. 5, the photocurrent increases as the concentration of the loaded sample increases, and the resultant equation is Y=1.6×10⁻⁶+1.38×10⁻⁸X in which Y represents the photocurrent in a unit of ampere (A) and X represents the HSA concentration in a unit of μg/mL. The result indicates that the photocurrent increases 13.8 nA as the HSA concentration increases 1 μg/mL.

If the semiconductor device analyzer 60 is connected to a computation module where the result of linear regression has been input, the computation module can directly demonstrate the resultant concentration of a tested sample with an unknown concentration during detection.

Although the present invention has been explained in relation to its preferred embodiment, it is to be understood that many other possible modifications and variations can be made without departing from the scope of the invention as hereinafter claimed.

What is claimed is:

1. A fluorescence detection system for measuring biomolecules comprising:
a fluorescence detection device comprising a substrate and plural phototransistors arranged on the substrate, wherein each phototransistor comprises an emitter, a collector locating on the substrate, and a base between the emitter and the collector, and a base-collector diode junction functions as an absorber to convert fluorescence to photocurrent;
a light source exciting a fluorescent dye contained in a biomolecule sample;
a sample-loading unit loading or transporting the biomolecule sample containing the excited fluorescent dye onto a sensing zone of the fluorescence detection device; and
an analysis-reading device measuring photocurrent output from the fluorescence detection device under a bias.

2. The fluorescence detection system as claimed in claim 1, wherein the analysis-reading device further comprises a computation module calculating a biomolecule content of the biomolecule sample from the photocurrent.

3. The fluorescence detection system as claimed in claim 1, wherein an area of the emitter is smaller than that of the base in the phototransistors of the fluorescence detection device.

4. The fluorescence detection system as claimed in claim 1, wherein the phototransistors are connected in parallel in the fluorescence detection device.

5. The fluorescence detection system as claimed in claim 1, wherein a material system of the emitter, the collector, and the base in the phototransistors of the fluorescence detection device is selected from at least one in the group consisting of AlGaAs/GaAs, InGaP/GaAs, AlInAs/InGaAs/InP, InP/InGaAs, InP/GaAsSb/InP, AlInAs/GaAsSb/InP, Si/SiGe, and GaN/SiC.

6. The fluorescence detection system as claimed in claim 1, wherein the light source excites the fluorescent dye into an excited state.

7. The fluorescence detection system as claimed in claim 1, wherein the biomolecule is selected from the group consisting of nucleic acid, carbohydrate, protein, lipid, phospholipid, glycolipid, sterol, vitamin, hormone, amino acid, nucleotide, and peptide.

8. A fluorescence detection method, comprising the following steps:
illuminating a biomolecule sample containing a fluorescent dye by a light source;
detecting the biomolecule sample by a fluorescence detection device under a bias, wherein the fluorescence detection device comprises a substrate and plural phototransistors arranged on the substrate, and each phototransistor comprises an emitter, a collector locating on the substrate, and a base between the emitter and the collector, wherein a base-collector diode junction functions as an absorber to convert fluorescence to photocurrent; and
measuring photocurrent output from the fluorescence detection device.

9. The fluorescence detection method as claimed in claim 8, further comprising the following step: converting the photocurrent into a biomolecule content of the biomolecule sample based on a current-content standard curve.

10. The fluorescence detection method as claimed in claim 8, wherein an area of the emitter is smaller than that of the base in the phototransistors of the fluorescence detection device.

11. The fluorescence detection method as claimed in claim 8, wherein the phototransistors are connected in parallel in the fluorescence detection device.

12. The fluorescence detection method as claimed in claim 8, wherein a material system of the emitter, the collector, and the base in the phototransistors of the fluorescence detection device is selected from at least one in the group consisting of AlGaAs/GaAs, InGaP/GaAs, AlInAs/InGaAs/InP, InP/InGaAs, InP/GaAsSb/InP, AlInAs/GaAsSb/InP, Si/SiGe, and GaN/SiC.

13. The fluorescence detection method as claimed in claim 8, wherein the light source excites the fluorescent dye into an excited state.

14. The fluorescence detection method as claimed in claim 8, wherein the biomolecule is selected from the group consisting of nucleic acid, carbohydrate, protein, lipid, phospholipid, glycolipid, sterol, vitamin, hormone, amino acid, nucleotide, and peptide.

15. A fluorescence detection device for measuring biomolecules comprising:
a substrate; and
plural phototransistors arranged on the substrate, wherein each phototransistor comprises an emitter, a collector locating on the substrate, and a base between the emitter and the collector, and a base-collector diode junction functions as an absorber to convert fluorescence to photocurrent.

16. The fluorescence detection device as claimed in claim 15, wherein an area of the emitter is smaller than that of the base in the phototransistors.

17. The fluorescence detection device as claimed in claim 15, wherein the phototransistors are connected in parallel.

18. The fluorescence detection device as claimed in claim 15, wherein a material system of the emitter, the collector, and the base in the phototransistors is selected from at least one in the group consisting of AlGaAs/GaAs, InGaP/GaAs, AlInAs/InGaAs/InP, InP/InGaAs, InP/GaAsSb/InP, AlInAs/GaAsSb/InP, Si/SiGe, and GaN/SiC.
